(19) United States
(12) Patent Application Publication
(10) Pub. No.: US 2021/0209398 A1
(43) Pub. Date: Jul. 8, 2021

(54) MEDICAL IMAGE PROCESSING APPARATUS, PROCESSOR DEVICE, MEDICAL IMAGE PROCESSING METHOD, AND PROGRAM

(71) Applicant: FUJIFILM Corporation, Tokyo (JP)

(72) Inventor: Maiko ENDO, Kanagawa (JP)

(73) Assignee: FUJIFILM Corporation, Tokyo (JP)

(21) Appl. No.: 17/206,140

(22) Filed: Mar. 19, 2021

Related U.S. Application Data
(63) Continuation of application No. PCT/JP2019/037477, filed on Sep. 25, 2019.

(30) Foreign Application Priority Data
Sep. 26, 2018 (JP) 2018-180301

(51) Int. Cl. G06K 9/32; G06T 5/50; G06T 7/00; H04N 7/18; G06F 3/14; A61B 1/00

(52) U.S. Cl. CPC G06K 9/3233; G06T 5/50; G06T 7/0012; H04N 7/18; G06F 3/14; H04N 2005/2255; A61B 1/0005; G06T 2207/20221; G06T 2207/30004; G06K 2209/05; G06T 2207/10068; A61B 1/00006

(57) ABSTRACT

Provided are a medical image processing apparatus, a processor device, a medical image processing method, and a program that may suppress hindrance of visual recognition of a medical image when a region of interest in the medical image is reported. A medical image processing apparatus includes an image acquisition unit (40) that acquires an endoscopic image (38), a region-of-interest detection unit (41) that detects regions of interest, an emphasis candidate region setting unit (42a) that sets, for each of the regions of interest, an emphasis candidate region that is a candidate for an emphasis region for emphasizing the region of interest when the endoscopic image is displayed using a monitor device (16), an emphasis region adjustment unit (42b) that sets, in a case where two or more regions of interest are detected, the emphasis region obtained by merging the emphasis candidate regions respectively corresponding to the two or more regions of interest in accordance with a distance between the two or more regions of interest, and a display control unit (44) that causes the monitor device to display the emphasis region.

### CROSS-REFERENCE TO RELATED APPLICATIONS

The present application is a Continuation of PCT International Application No. PCT/JP2019/037477 filed on Sep. 25, 2019 claiming priority under 35 U.S.C § 119(a) to Japanese Patent Application No. 2018-180301 filed on Sep. 26, 2018. Each of the above applications is hereby expressly incorporated by reference, in its entirety, into the present application.

### BACKGROUND OF THE INVENTION

### 1. Field of the Invention

The present invention relates to a medical image processing apparatus, a processor device, a medical image processing method, and a program, and more particularly to reporting of a detection result.

### 2. Description of the Related Art

JP5693550B describes an image processing apparatus that, when reading out an encoded X-ray medical image from a storage medium, automatically determines a region useful for diagnosis and preferentially reads out and displays data relating to the region.

When a plurality of shadow patterns determined to be positive are close to each other, the apparatus described in JP5693550B sets the plurality of shadow patterns as a single positive region. The device described in JP5693550B also converts the positive region to have a rectangular shape to specify an encoded region.

JP5658873B describes an endoscope system that detects a candidate region of interest from a special-light image, calculates reliability indicating a likelihood of the candidate region of interest being a region of interest, sets a region of interest on the basis of the reliability, and performs processing on a corresponding region of interest in a normal-light image. The system described in JP5658873B sets an alert region on the basis of a region of interest and a priority indicating a degree at which the alert region is to be preferentially displayed.

When the number of alert regions is predicted to exceed an upper limit, the system described in JP5658873B does not display the number of alert regions that exceeds the upper limit so as to suppress a situation in which too many alert regions are displayed for a doctor to recognize at one time.

JP2011-255006A describes an image processing apparatus that determines whether to display an alert image corresponding to a region of interest in accordance with a detection result of the region of interest, and displays the alert image corresponding to a display-target region of interest that is a region of interest for which the alert image is determined to be displayed.

In the invention described in JP2011-255006A, when the number of regions of interest is small, an alert image is not displayed for a region of interest having a size exceeding a threshold value. When the number of regions of interest is large, an alert image is not displayed for a region of interest having a size that is smaller than or equal to the threshold value.

### SUMMARY OF THE INVENTION

When a plurality of lesions are detected in a medical diagnosis system that automatically detects a lesion portion, a plurality of reporting portions for reporting lesion detected locations may be present for the respective lesion detected locations. When many lesions are detected and many reporting portions are present, a display screen of a medical image becomes complicated. This may hinder a doctor from making a diagnosis.

The invention described in JP5693550B handles, as a single positive region, a plurality of positive regions located within a range that is consequently handled as a single positive region, and thus does not focus on and overcome an issue that the presence of many reporting portions for reporting positive regions hinders visual recognition of lesions.

The invention described in JP5658873B does not display the number of alert regions that exceeds the upper limit. Thus, an alert region that is to be displayed may not be displayed.

The invention described in JP2011-255006A does not display an alert image in accordance with the number of regions of interest and the size of a region of interest. Thus, an alert region that is to be displayed may not be displayed.

The present invention is made in view of such circumstances, and an object thereof is to provide a medical image processing apparatus, a processor device, a medical image processing method, and a program that may suppress hindrance of visual recognition of a medical image when a region of interest in the medical image is reported.

In order to achieve the above object, the following aspects of the invention are provided.

A medical image processing apparatus according to a first aspect is a medical image processing apparatus including an image acquisition unit that acquires a medical image; a region-of-interest detection unit that detects regions of interest from the medical image; an emphasis candidate region setting unit that sets, for each of the regions of interest, an emphasis candidate region that is a candidate for an emphasis region for emphasizing the region of interest when the medical image is displayed using a display device; an emphasis region adjustment unit that sets, in a case where two or more regions of interest are detected, the emphasis region obtained by merging the emphasis candidate regions respectively corresponding to the two or more regions of interest in accordance with a distance between the two or more regions of interest; and a display control unit that causes the display device to display the emphasis region.

According to the first aspect, in the case where two or more regions of interest are detected, an emphasis region is set which is obtained by merging emphasis candidate regions respectively corresponding to the two or more regions of interest in accordance with a distance between the two or more regions of interest. This reduces the number of emphasis regions for emphasizing the regions of interest and thus may suppress hindrance of visual recognition of a medical image.

The emphasis candidate adjustment unit may merge the emphasis candidate regions in accordance with the distance between the two or more regions of interest, or may merge the emphasis candidate regions in accordance with a physical quantity that changes on the basis of the distance between the two or more regions of interest.

In a second aspect, in the medical image processing apparatus according to the first aspect, the image acquisition unit may acquire a still image as the medical image, and in a case where two or more regions of interest are detected in the still image, the emphasis region adjustment unit may merge the emphasis candidate regions respectively corresponding to the two or more regions of interest in accordance with a distance between the two or more regions of interest.

The still image may include each frame image constituting the moving image.

In a third aspect, in the medical image processing apparatus according to the first aspect, the image acquisition unit may acquire a moving image as the medical image, and in a case where two or more regions of interest are detected in two or more different frame images constituting the moving image, the emphasis region adjustment unit may merge the emphasis candidate regions respectively corresponding to the two or more regions of interest in accordance with a distance between the two or more regions of interest.

The case where two or more regions of interest are detected in different frame images of a moving image may include a case where a first region of interest is detected in a first frame image and a second region of interest is detected in a second frame image subsequent to the first frame image.

In a fourth aspect, in the medical image processing apparatus according to the third aspect, in a case where a region of interest detected as a single region of interest in a first frame image is detected as two or more regions of interest in a second frame image subsequent to the first frame image, the emphasis region adjustment unit may merge the emphasis candidate regions respectively corresponding to the two or more regions of interest.

The emphasis region adjustment unit may determine whether to merge the emphasis candidate region of the first frame image and the emphasis candidate region of the second frame image on the basis of a feature quantity of the region of interest of the first frame image and a feature quantity of the region of interest of the second frame image.

In a fifth aspect, the medical image processing apparatus according to the second or third aspect, the emphasis region adjustment unit may merge the emphasis candidate regions respectively corresponding to the two or more regions of interest in a case where the distance between the two or more regions of interest is greater than 0 and is less than or equal to a predetermined threshold value.

In a sixth aspect, in the medical image processing apparatus according to any one of the first to fifth aspects, wherein the emphasis region adjustment unit may merge the emphasis candidate regions respectively corresponding to the two or more regions of interest in accordance with a distance between centers of gravity of the two or more regions of interest.

In a seventh aspect, in the medical image processing apparatus according to any one of the first to fifth aspects, the emphasis region adjustment unit may merge the emphasis candidate regions respectively corresponding to the two or more regions of interest in accordance with a degree of overlap of the emphasis candidate regions respectively corresponding to the two or more regions of interest.

When an area of overlap of the emphasis candidate regions is used as the degree of overlap of the emphasis candidate regions, “the area of overlap of the emphasis candidate regions being greater than or equal to a predetermined area threshold value” is equivalent to “the distance between the regions of interest being greater than 0 and being less than or equal to a predetermined threshold value”.

In an eighth aspect, in the medical image processing apparatus according to any one of the first to seventh aspects, the emphasis region adjustment unit may merge the emphasis candidate regions respectively corresponding to the two or more regions of interest in accordance with feature quantities of the regions of interest.

In a ninth aspect, in the medical image processing apparatus according to any one of the first to eighth aspects, the emphasis region adjustment unit may set the emphasis region that includes all of the two or more regions of interest to be merged.

A processor device according to a tenth aspect is a processor device including an endoscope control unit that controls an endoscope apparatus; an image acquisition unit that acquires a medical image from the endoscope apparatus; a region-of-interest detection unit that detects regions of interest from the medical image; an emphasis candidate region setting unit that sets, for each of the regions of interest, an emphasis candidate region that is a candidate for an emphasis region for emphasizing the region of interest when the medical image is displayed using a display device; an emphasis region adjustment unit that sets, in a case where two or more regions of interest are detected, the emphasis region obtained by merging the emphasis candidate regions respectively corresponding to the two or more regions of interest in accordance with a distance between the two or more regions of interest; and a display control unit that causes the display device to display the emphasis region.

According to the tenth aspect, substantially the same advantage as that of the first aspect can be obtained.

The tenth aspect may be appropriately combined with any of features that are substantially the same as those specified in the second to ninth aspects. In such a case, a constituent element responsible for a process or function specified in the medical image processing apparatus can be grasped as a constituent element responsible for the corresponding process or function in the processor device.

A medical image processing method according to an eleventh aspect is a medical image processing method including an image acquisition step of acquiring a medical image; a region-of-interest detection step of detecting regions of interest from the medical image; an emphasis candidate region setting step of setting, for each of the regions of interest, an emphasis candidate region that is a candidate for an emphasis region for emphasizing the region of interest when the medical image is displayed using a display device; an emphasis region adjustment step of setting, in a case where two or more regions of interest are detected, the emphasis region obtained by merging the emphasis candidate regions respectively corresponding to the two or more regions of interest in accordance with a distance between the two or more regions of interest; and a display control step of causing the display device to display the emphasis region.

According to the eleventh aspect, substantially the same advantage as that of the first aspect can be obtained.

The eleventh aspect may be appropriately combined with any of features that are substantially the same as those specified in the second to ninth aspects. In such a case, a constituent element responsible for a process or function specified in the medical image processing apparatus can be grasped as a constituent element responsible for the corresponding process or function in the medical image processing method.

A program according to a twelfth aspect is a program that causes a computer to implement an image acquisition function of acquiring a medical image; a region-of-interest detection function of detecting regions of interest from the medical image; an emphasis candidate region setting function of setting, for each of the regions of interest, an emphasis candidate region that is a candidate for an emphasis region for emphasizing the region of interest when the medical image is displayed using a display device; an emphasis region adjustment function of setting, in a case where two or more regions of interest are detected, the emphasis region obtained by merging the emphasis candidate regions respectively corresponding to the two or more regions of interest in accordance with a distance between the two or more regions of interest; and a display control function of causing the display device to display the emphasis region.

According to the twelfth aspect, substantially the same advantage as that of the first aspect can be obtained.

The twelfth aspect may be appropriately combined with any of features that are substantially the same as those specified in the second to ninth aspects. In such a case, a constituent element responsible for a process or function specified in the medical image processing apparatus can be grasped as a constituent element responsible for the corresponding process or function in the program.

According to the present invention, when two or more regions of interest are detected, an emphasis region is set which is obtained by merging emphasis candidate regions respectively corresponding to the two or more regions of interest in accordance with a distance between the two or more regions of interest. This reduces the number of emphasis regions for emphasizing the regions of interest and thus may suppress hindrance of visual recognition of a medical image.

### BRIEF DESCRIPTION OF THE DRAWINGS

FIG. 1 is an overall configuration diagram of an endoscope system including a medical image processing apparatus according to embodiments;

FIG. 2 is a block diagram illustrating a hardware configuration of the medical image processing apparatus;

FIG. 3 is a functional block diagram of the medical image processing apparatus according to a first embodiment;

FIG. 4 is an explanatory diagram of detection of regions of interest and setting of emphasis candidate regions;

FIG. 5 is an explanatory diagram of derivation of a center-of-gravity distance between the regions of interest;

FIG. 6 is an explanatory diagram of merging of emphasis candidate regions on the basis of the center-of-gravity distance;

FIG. 7 is a flowchart of a medical image processing method according to the first embodiment;

FIG. 8 is an explanatory diagram of derivation of a degree of overlap of emphasis candidate regions;

FIG. 9 is an explanatory diagram of merging of emphasis candidate regions on the basis of the degree of overlap;

FIG. 10 is an explanatory diagram of merging of emphasis candidate regions applied to a medical image processing apparatus according to a third embodiment;

FIG. 11 is an explanatory diagram of derivation of a distance between regions of interest;

FIG. 12 is an explanatory diagram of merging of emphasis candidate regions on the basis of the distance between regions of interest;

FIG. 13 is a schematic diagram of merging of a plurality of emphasis candidate regions on the basis of feature quantities of regions of interest; and

FIG. 14 is a schematic diagram of another example of merging of a plurality of emphasis candidate regions on the basis of feature quantities of regions of interest.

### DESCRIPTION OF THE PREFERRED EMBODIMENTS

Preferred embodiments of the present invention will be described in detail below with reference to the accompanying drawings. The same constituent elements are denoted by the same reference signs herein, and redundant description will be appropriately omitted.

Overall Configuration of Endoscope System

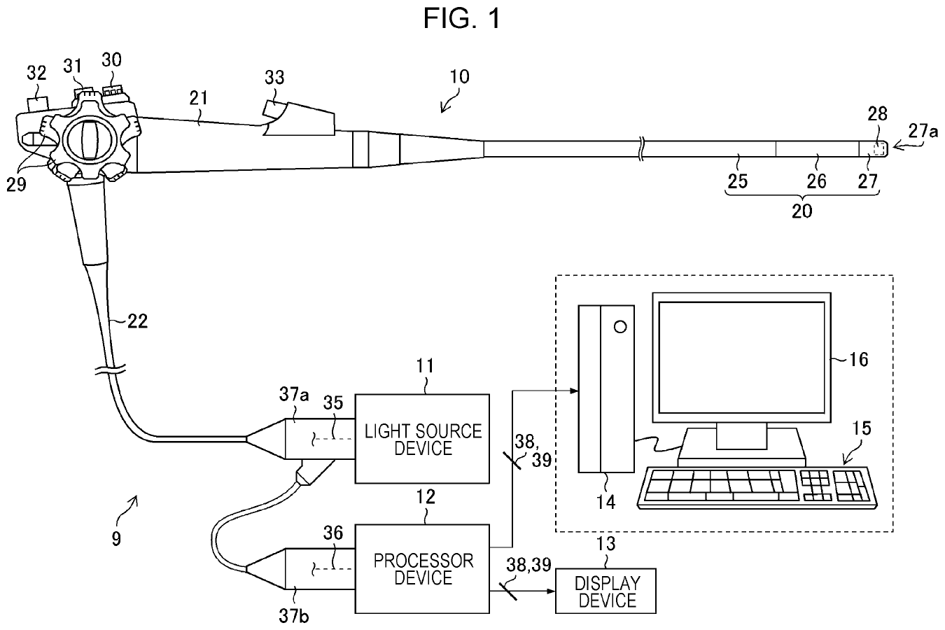

FIG. 1 is an overall configuration diagram of an endoscope system including a medical image processing apparatus according to embodiments. An endoscope system 9 illustrated in FIG. 1 includes an endoscope 10, a light source device 11, a processor device 12, a display device 13, a medical image processing apparatus 14, an input device 15, and a monitor device 16. The endoscope system 9 described in the embodiments is an example of an endoscope apparatus.

The endoscope 10 illustrated in FIG. 1 is an electronic endoscope and is also a flexible endoscope. The endoscope 10 includes an insertion section 20, an operation section 21, and a universal cord 22. The insertion section 20 is inserted into a subject. The entire insertion section 20 is formed to have an elongated shape with a small diameter.

The insertion section 20 includes a soft part 25, a bending part 26, and a tip part 27. The soft part 25, the bending part 26, and the tip part 27 are coupled to each other to constitute the insertion section 20. The soft part 25 has flexibility sequentially from a proximal end side to a distal end side of the insertion section 20. The bending part 26 has a structure that is bendable when the operation section 21 is operated. The tip part 27 includes an imaging optical system (not illustrated), an imaging element 28, and so on.

A CMOS imaging element or a CCD imaging element is used as the imaging element 28. CMOS is an abbreviation for complementary metal oxide semiconductor. CCD is an abbreviation for charge coupled device.

An observation window (not illustrated) is disposed on a tip surface 27a of the tip part 27. The observation window is an opening formed on the tip surface 27a of the tip part 27. A cover (not illustrated) is attached to the observation window. The imaging optical system (not illustrated) is disposed behind the observation window. Image light of a site to be observed is incident onto an imaging surface of the imaging element 28 through the observation window, the imaging optical system, and so on. The imaging element 28 images the image light of the site to be observed incident onto the imaging surface of the imaging element 28 and outputs an imaging signal. The term “imaging” used herein includes the meaning of converting light reflected off from a site to be observed into an electric signal.

The operation section 21 is coupled to the proximal end side of the insertion section 20. The operation section 21 includes various operating members to be operated by a technician. Specifically, the operation section 21 includes two types of bending operation knobs 29. The bending operation knobs 29 are used to perform an operation of bending the bending part 26. Note that the technician may also be referred to as a doctor, an operator, an observer, a user, or the like.

The operation section 21 includes an air/water supply button 30 and a suction button 31. The air/water supply button 30 is used when the technician performs an air/water supply operation. The suction button 31 is used when the technician performs a suction operation.

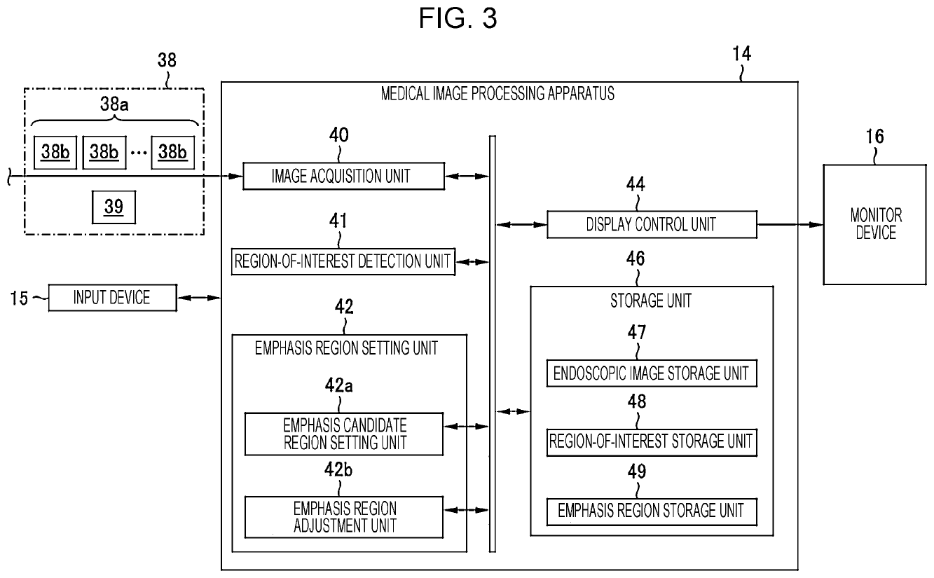

The operation section 21 includes a still image capturing instruction part 32 and a treatment tool introduction port 33. The still image capturing instruction part 32 is operated by the technician when a still image of the site to be observed is captured. The treatment tool introduction port 33 is an opening through which a treatment tool is to be inserted into a treatment tool insertion path that is inserted inside the insertion section 20. Note that illustration of the treatment tool insertion path and the treatment tool is omitted. A still image, assigned a reference sign 39, is illustrated in FIG. 3.

The universal cord 22 is a connection cord that connects the endoscope 10 to the light source device 11. The universal cord 22 includes therein a light guide 35, a signal cable 36, and a fluid tube (not illustrated), which are inserted inside the insertion section 20.

In addition, a tip part of the universal cord 22 includes a connector 37a to be connected to the light source device 11 and a connector 37b branching from the connector 37a and to be connected to the processor device 12.

When the connector 37a is connected to the light source device 11, the light guide 35 and the fluid tube (not illustrated) are inserted into the light source device 11. Consequently, necessary illumination light, water, and gas are supplied from the light source device 11 to the endoscope 10 through the light guide 35 and the fluid tube (not illustrated).

As a result, the illumination light is radiated from an illumination window (not illustrated) of the tip surface 27a of the tip part 27 toward the site to be observed. In addition, in response to an operation of pressing the air/water supply button 30, gas or water is ejected from an air/water supply nozzle (not illustrated) of the tip surface 27a of the tip part 27 toward the observation window (not illustrated) of the tip surface 27a.

When the connector 37b is connected to the processor device 12, the signal cable 36 and the processor device 12 are electrically connected to each other. Consequently, an imaging signal of the site to be observed is output from the imaging element 28 of the endoscope 10 to the processor device 12 through the signal cable 36. Also, a control signal is output from the processor device 12 to the endoscope 10 through the signal cable 36.

In the present embodiments, the flexible endoscope is described as an example of the endoscope 10. However, various types of electronic endoscopes capable of capturing a moving image of a site to be observed, such as a rigid endoscope, may be used as the endoscope 10.

The light source device 11 supplies illumination light to the light guide 35 of the endoscope 10 through the connector 37a. White light or light in a specific wavelength range is usable as the illumination light. As the illumination light, white light and light in a specific wavelength range may be used in combination. The light source device 11 is configured to be able to appropriately select, as the illumination light, light in a wavelength range corresponding to an observation purpose.

The white light may be light in a white wavelength range or light in a plurality of wavelength ranges. The specific wavelength range is a range narrower than the white wavelength range. As the light in the specific wavelength range, light in a single wavelength range may be used, or light in a plurality of wavelength ranges may be used. Light in the specific wavelength range may be referred to as special light.

The processor device 12 controls the operation of the endoscope 10 through the connector 37b and the signal cable 36. The processor device 12 also acquires an imaging signal from the imaging element 28 of the endoscope 10 through the connector 37b and the signal cable 36. The processor device 12 uses a predetermined frame rate to acquire an imaging signal output from the endoscope 10.

The processor device 12 generates an endoscopic image 38, which is an observation image of the site to be observed, on the basis of the imaging signal acquired from the endoscope 10. Herein, the endoscopic image 38 includes a moving image. The endoscopic image 38 may include the still image 39. Note that a moving image, assigned a reference sign 38a, is illustrated in FIG. 3. The endoscopic image 38 in the embodiments is an example of a medical image.

When the still image capturing instruction part 32 of the operation section 21 is operated, the processor device 12 generates the still image 39 of the site to be observed on the basis of the imaging signal acquired from the imaging element 28 in parallel with generation of the moving image. The still image 39 may be generated to have a resolution higher than the resolution of the moving image.

When generating the endoscopic image 38, the processor device 12 performs image quality correction in which digital signal processing such as white balance adjustment and shading correction are used. The processor device 12 may add accessory information defined by the DICOM standard to the endoscopic image 38. Note that DICOM is an abbreviation for Digital Imaging and Communications in Medicine. The processor device 12 described in the embodiments is an example of a processor device including an endoscope control unit that controls the endoscope system 9.

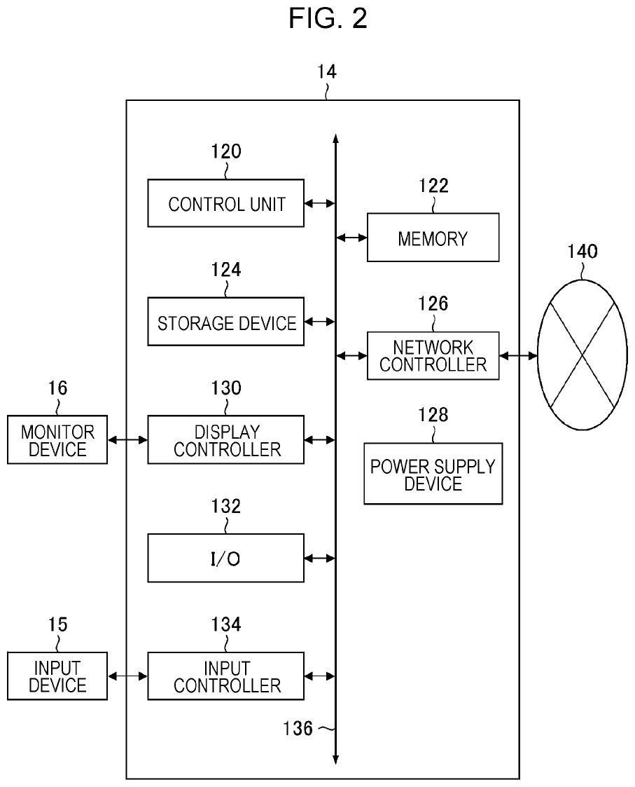

The processor device 12 outputs the endoscopic image 38 to each of the display device 13 and the medical image processing apparatus 14. The processor device 12 may output the endoscopic image 38 to a storage device (not illustrated) via a network (not illustrated) in accordance with a communication protocol compliant with the DICOM standard. Note that a network 140 illustrated in FIG. 2 may be used as the network.

The display device 13 is connected to the processor device 12. The display device 13 displays the endoscopic image 38 transmitted from the processor device 12. The technician may perform an operation of moving the insertion section 20 forward and backward while checking the endoscopic image 38 displayed on the display device 13. Upon detecting a lesion or the like at the site to be observed, the technician may operate the still image capturing instruction part 32 to capture a still image of the site to be observed.

A computer is used as the medical image processing apparatus 14. A keyboard, a mouse, and the like connectable to the computer are used as the input device 15. The input device 15 and the computer may be connected to each other either with a cable or wirelessly. Various monitors connectable to the computer are used as the monitor device 16.

As the medical image processing apparatus 14, a diagnosis assistant apparatus such as a workstation or a server apparatus may be used. In this case, the input device 15 and the monitor device 16 are provided for each of a plurality of terminals connected to the workstation or the like. Further, as the medical image processing apparatus 14, a medical service assistant apparatus that assists creation of a medical report or the like may be used.

The medical image processing apparatus 14 acquires the endoscopic image 38 and stores the endoscopic image 38. The medical image processing apparatus 14 controls reproduction performed by the monitor device 16. Note that the term “image” used herein includes a meaning of an electric signal representing the image and a meaning of image data such as information representing the image. The term “image” used herein means at least any of an image itself or image data.

Further, the term “storing an image” can be interpreted as “saving an image”, “storage of an image”, or the like. “Storing an image” used herein means “storing an image in a non-transitory manner”. The medical image processing apparatus 14 may include a temporary storage memory that temporarily stores an image.

The input device 15 is used to input an operation instruction for the medical image processing apparatus 14. The monitor device 16 displays the endoscopic image 38 under the control of the medical image processing apparatus 14. The monitor device 16 may function as a display unit of various kinds of information in the medical image processing apparatus 14.

The medical image processing apparatus 14 may be connected to a storage device (not illustrated) via a network (not illustrated in FIG. 1). The DICOM standard, a protocol compliant with the DICOM standard, and the like may be used as the image storage format and for the communication between apparatuses via the network.

As the storage device (not illustrated), a storage or the like that stores data in a non-transitory manner may be used. The storage device may be managed using a server apparatus (not illustrated). As the server apparatus, a computer that stores and manages various kinds of data may be used.

### Hardware Configuration of Medical Image Processing Apparatus

FIG. 2 is a block diagram illustrating a hardware configuration of the medical image processing apparatus. The medical image processing apparatus 14 illustrated in FIG. 2 includes a control unit 120, a memory 122, a storage device 124, a network controller 126, a power supply device 128, a display controller 130, an input/output interface 132, and an input controller 134. Note that I/O illustrated in FIG. 2 represents the input/output interface 132.

The control unit 120, the memory 122, the storage device 124, the network controller 126, the display controller 130, and the input/output interface 132 are connected to each other via a bus 136 so that data communication can be performed therebetween.

### Control Unit

The control unit 120 functions as an overall control unit, various calculation units, and a storage control unit of the medical image processing apparatus 14. The control unit 120 executes a program stored in a read-only memory (ROM) included in the memory 122.

The control unit 120 may download a program from an external storage device (not illustrated) via the network controller 126 and execute the downloaded program. The external storage device may be communicably connected to the medical image processing apparatus 14 via the network 140.

The control unit 120 uses, as a calculation area, a random access memory (RAM) included in the memory 122 and executes various processes in cooperation with various programs. Consequently, various functions of the medical image processing apparatus 14 are implemented.

The control unit 120 controls reading out of data from the storage device 124 and writing of data to the storage device 124. The control unit 120 may acquire various kinds of data from an external storage device via the network controller 126. The control unit 120 is capable of executing various processes such as calculations using the acquired various kinds of data.

The control unit 120 may include one processor or two or more processors. Examples of the processor include a field programmable gate array (FPGA), a programmable logic device (PLD), and so on. An FPGA and a PLD are devices whose circuit configurations are changeable after being manufactured.

Another example of the processor is an application-specific integrated circuit (ASIC). An ASIC includes a circuit configuration dedicatedly designed to execute specific processing.

The control unit 120 may use two or more processors of the same kind. For example, the control unit 120 may use two or more FPGAs or two PLDs. The control unit 120 may use two or more processors of different kinds. For example, the control unit 120 may use one or more FPGAs and one or more ASICs.

When the medical image processing apparatus 14 includes a plurality of control units 120, the plurality of control units 120 may be configured using a single processor. As an example of configuring the plurality of control units 120 using a single processor, there is a form in which the single processor is configured using a combination of one or more central processing units (CPUs) and software and this processor functions as the plurality of control units 120. Note that software used herein is synonymous with a program.

As another example of configuring the plurality of control units 120 using a single processor, there is a form in which a processor that implements, with a single IC chip, the functions of the entire system including the plurality of control units 120. Representative examples of the processor that implements, with a single IC chip, the functions of the entire system including the plurality of control units 120 include a system on a chip (SoC). Note that IC is an abbreviation for integrated circuit.

As described above, the control unit 120 is configured using one or more of various processors as the hardware structure.

### Memory

The memory 122 includes a ROM (not illustrated) and a RAM (not illustrated). The ROM stores various programs to be executed in the medical image processing apparatus 14. The ROM stores parameters, files, and the like used for executing various programs. The RAM functions as a temporary data storage area, a work area for the control unit 120, and the like.

### Storage Device

The storage device 124 stores various kinds of data in a non-transitory manner. The storage device 124 may be externally attached to the medical image processing apparatus 14. Instead of or along with the storage device 124, a large-capacity semiconductor memory device may be used.

### Network Controller

The network controller 126 controls data communication between the medical image processing apparatus 14 and an external apparatus. The control of the data communication may include management of the traffic in the data communication. As the network 140 to which the medical image processing apparatus 14 is connected via the network controller 126, a known network such as a local area network (LAN) may be used.

### Power Supply Device

As the power supply device 128, a large-capacity power supply device such as an uninterruptible power supply (UPS) is used. The power supply device 128 supplies power to each unit of the medical image processing apparatus 14 when the commercial power supply is cut off due to a power failure or the like.

### Display Controller

The display controller 130 functions as a display driver that controls the monitor device 16 on the basis of an instruction signal transmitted from the control unit 120.

### Input/Output Interface

The input/output interface 132 communicably connects the medical image processing apparatus 14 and an external device to each other. A communication standard such as Universal Serial Bus (USB) may be used for the input/output interface 132.

### Input Controller

The input controller 134 converts the format of a signal input using the input device 15 into a format suitable for processing performed by the medical image processing apparatus 14. Information input from the input device 15 via the input controller 134 is transmitted to each unit via the control unit 120.

Note that the hardware configuration of the medical image processing apparatus 14 illustrated in FIG. 2 is merely an example. Thus, addition, deletion, and modification may be appropriately made. The hardware configuration of the medical image processing apparatus 14 illustrated in FIG. 2 is applicable to each of embodiments and modifications described below.

### Medical Image Processing Apparatus According to First Embodiment Description of Functional Blocks

FIG. 3 is a functional block diagram of the medical image processing apparatus according to the first embodiment. The medical image processing apparatus 14 includes an image acquisition unit 40, a region-of-interest detection unit 41, an emphasis region setting unit 42, a display control unit 44, and a storage unit 46.

The image acquisition unit 40 acquires the endoscopic image 38 from the processor device 12. The image acquisition unit 40 stores the endoscopic image 38 in an endoscopic image storage unit 47.

The image acquisition unit 40 may acquire the endoscopic image 38 from the processor device 12 via an information storage medium such as a memory card. The image acquisition unit 40 may acquire the endoscopic image 38 via the network 140 illustrated in FIG. 2.

The image acquisition unit 40 may acquire the moving image 38a constituted by time-series frame images 38b. The image acquisition unit 40 may acquire the still image 39 in the case where still image capturing is performed during capturing of the moving image 38a.

The region-of-interest detection unit 41 detects a region of interest from the endoscopic image 38. The region-of-interest detection unit 41 may divide the frame image 38b constituting the endoscopic image 38 into a plurality of local regions, calculate feature quantities for the respective local regions, and detect a region of interest on the basis of the feature quantities for the respective local regions.

The emphasis region setting unit 42 sets an emphasis region for emphasizing the region of interest detected from the endoscopic image 38. The emphasis region setting unit 42 includes an emphasis candidate region setting unit 42a and an emphasis region adjustment unit 42b.

The emphasis candidate region setting unit 42a sets an emphasis candidate region which is a candidate for an emphasis region. When a plurality of regions of interest are set, the emphasis candidate region setting unit 42a sets an emphasis candidate region for each of the regions of interest. The emphasis candidate region setting unit 42a may set emphasis candidate regions for all the endoscopic images 38 in which the region of interest is detected. Note that an emphasis candidate region is not an object displayed on the display screen of the endoscopic image 38 but is an object that is used in calculation but is not displayed on the display screen.

The emphasis candidate region setting unit 42a may set the location of the emphasis candidate region on the basis of coordinate values of the region of interest. The emphasis candidate region setting unit 42a may acquire, as the coordinate values of the region of interest, coordinate values of the center of gravity of the region of interest. The emphasis candidate region setting unit 42a may acquire, as the coordinate values of the region of interest, coordinate values on a closed curve constituting an edge of the region of interest. The emphasis candidate region setting unit 42a may set, as an outer shape of the emphasis candidate region, a quadrangle in which the closed curve constituting the edge of the region of interest is inscribed. Instead of the quadrangle, a circle or a polygonal shape other than a quadrangle may be used.

When a plurality of emphasis candidate regions are set, the emphasis region adjustment unit 42b merges the plurality of emphasis candidate regions in accordance with a distance between regions of interest to set a single emphasis region for the plurality of regions of interest. The emphasis region adjustment unit 42b sets, as an emphasis region, an emphasis candidate region that is not merged with another emphasis candidate region.

Although illustration is omitted, the medical image processing apparatus 14 may include a threshold value setting unit that sets a threshold value used for determining whether to merge a plurality of emphasis candidate regions. The threshold value setting unit may read out the threshold value from a threshold value storage unit in which the threshold value is stored in advance. The threshold value setting unit may set the threshold value on the basis of threshold value information input from the input device 15.

The display control unit 44 transmits, to the monitor device 16, a display control signal for displaying the endoscopic image 38 and the emphasis region on the monitor device 16. The display control unit 44 updates display of the endoscopic image 38 and display of the emphasis region using predetermined update intervals.

The monitor device 16 displays the endoscopic image 38 and the emphasis region. The monitor device 16 may display the emphasis region to be superimposed on the endoscopic image 38. The emphasis region is displayed so as not to hinder recognition of the endoscopic image 38.

The storage unit 46 includes the endoscopic image storage unit 47, a region-of-interest storage unit 48, and an emphasis region storage unit 49. The storage unit 46 may include the threshold value storage unit (not illustrated). The endoscopic image storage unit 47 stores the endoscopic image 38 acquired using the image acquisition unit 40.

The region-of-interest storage unit 48 stores information on the region of interest. The region-of-interest storage unit 48 may store information on the region of interest associated with the endoscopic image 38 in which the region of interest is detected. As the information on the region of interest, coordinate values of the region of interest in the endoscopic image 38 may be used. The coordinate values of the region of interest are the same as the coordinate values of the region of interest used when the emphasis region is set.

The emphasis region storage unit 49 stores information on the emphasis candidate region and the emphasis region. The emphasis region storage unit 49 may store information on the emphasis candidate region or the emphasis region associated with the endoscopic image 38 in which the emphasis candidate region or the emphasis region is set.

As the storage unit 46, one or more storage elements may be used. That is, the storage unit 46 may include three storage elements respectively corresponding to the endoscopic image storage unit 47, the region-of-interest storage unit 48, and the emphasis region storage unit 49. For each of the endoscopic image storage unit 47, the region-of-interest storage unit 48, and the emphasis region storage unit 49, a plurality of storage elements may be used. Further, two or all of the endoscopic image storage unit 47, the region-of-interest storage unit 48, and the emphasis region storage unit 49 may be constituted by a single storage element.

### Merging of Emphasis Candidate Regions

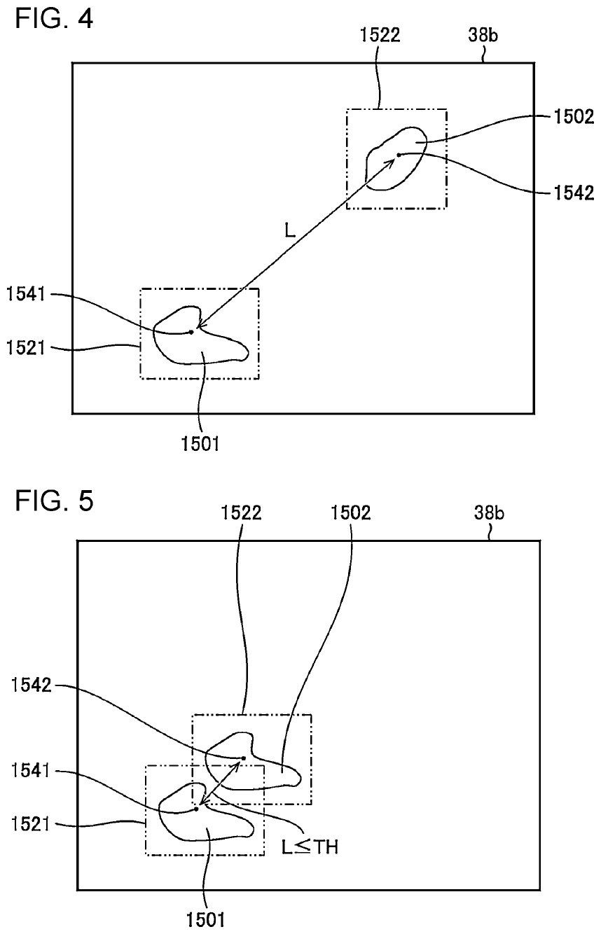

FIG. 4 is an explanatory diagram of detection of regions of interest and setting of emphasis candidate regions. The frame image 38b illustrated in FIG. 4 is any of frame images constituting the moving image 38a. In the following description, description of the frame image 38b is interchangeable with description of the still image 39. That is, the frame image 38b described in the embodiment is an example of a still image.

The region-of-interest detection unit 41 detects a first region of interest 1501 and a second region of interest 1502 from the frame image 38b illustrated in FIG. 4. The emphasis candidate region setting unit 42a sets a first emphasis candidate region 1521 for the first region of interest 1501 and sets a second emphasis candidate region 1522 for the second region of interest 1502.

The emphasis candidate region setting unit 42a acquires coordinate values of a center of gravity 1541 of the first region of interest 1501 and coordinate values of a closed curve representing an edge of the first region of interest 1501. The emphasis candidate region setting unit 42a sets the coordinate values of the center of gravity 1541 of the first region of interest 1501 as coordinate values of the first emphasis candidate region 1521.

On the basis of the coordinate values of the closed curve representing the edge of the first region of interest 1501, the emphasis candidate region setting unit 42a also sets a frame enclosing the closed curve representing the edge of the first region of interest 1501 as an outer shape of the first emphasis candidate region 1521.

The emphasis candidate region setting unit 42a sets coordinate values of a center of gravity 1542 of the second region of interest 1502 as coordinate value of a center of gravity of the second emphasis candidate region 1522. On the basis of the coordinate values of the closed curve representing the edge of the second region of interest 1502, the emphasis candidate region setting unit 42a sets a frame enclosing the closed curve representing the edge of the second region of interest 1502 as an outer shape of the second emphasis candidate region 1522. As the coordinate system used for the frame image 38b, a two-dimensional orthogonal coordinate system may be used.

The coordinate values is interchangeable with a pixel location. That is, when setting the first emphasis candidate region 1521, the emphasis candidate region setting unit 42a may identify a pixel location of the center of gravity 1541 of the first region of interest 1501 and pixel locations constituting the closed curve representing the edge of the first region of interest 1501. The same applies to the second emphasis candidate region 1522.

The emphasis region adjustment unit 42b determines the number of regions of interest in the frame image 38b. When a plurality of regions of interest are detected, the emphasis region adjustment unit 42b determines whether to merge emphasis candidate regions respectively set for the plurality of regions of interest in accordance with a distance between the plurality of regions of interest. As the distance between the plurality of regions of interest, a distance between centers of gravity of the regions of interest may be used.

That is, when the first region of interest 1501 and the second region of interest 1502 are detected from the frame image 38b, the emphasis region adjustment unit 42b acquires the coordinate values of the center of gravity 1541 of the first region of interest 1501 and the coordinate values of the center of gravity 1542 of the second region of interest 1502. The emphasis region adjustment unit 42b calculates a center-of-gravity distance L representing a distance between the center of gravity 1541 of the first region of interest 1501 and the center of gravity 1542 of the second region of interest 1502.

The emphasis region adjustment unit 42b determines whether to merge the first emphasis candidate region 1521 and the second emphasis candidate region 1522 on the basis of the center-of-gravity distance L between the regions of interest.

FIG. 5 is a schematic diagram of regions of interest and emphasis candidate regions when the emphasis candidate regions are merged. FIG. 5 illustrates the frame image 38b in which the center-of-gravity distance L between the center of gravity 1541 of the first region of interest 1501 and the center of gravity 1542 of the second region of interest 1502 is less than or equal to a predetermined threshold value TH. The expression “is less than or equal to the predetermined threshold value TH” used in the embodiments is an example of the expression “is less than or equal to a threshold value”.

If the center-of-gravity distance L between the first region of interest 1501 and the second region of interest 1502 is less than or equal to the predetermined threshold value TH, the emphasis region adjustment unit 42b merges the first emphasis candidate region 1521 and the second emphasis candidate region 1522.

On the other hand, if the center-of-gravity distance L between the first region of interest 1501 and the second region of interest 1502 exceeds the predetermined threshold value TH, the emphasis region adjustment unit 42b sets the first emphasis candidate region 1521 as an emphasis region of the first region of interest 1501. Similarly, the emphasis region adjustment unit 42b sets the second emphasis candidate region 1522 as an emphasis region of the second region of interest 1502.

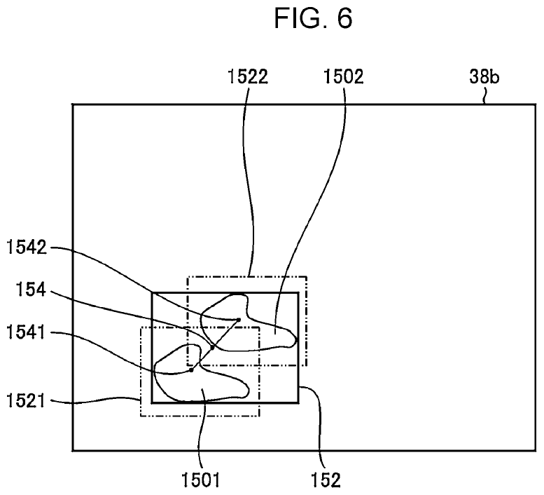

FIG. 6 is a schematic diagram of emphasis candidate regions and an emphasis region when the emphasis candidate regions are merged. The emphasis region adjustment unit 42b sets, for the first region of interest 1501 and the second region of interest 1502, an emphasis region 152 obtained by merging the first emphasis candidate region 1521 and the second emphasis candidate region 1522.

The emphasis region adjustment unit 42b calculates coordinate values of a center location between the coordinate values of the center of gravity 1541 of the first region of interest 1501 and the coordinate values of the center of gravity 1542 of the second region of interest 1502, and sets the calculated coordinate values as coordinate values of a center of gravity 154 of the emphasis region 152.

As the emphasis region 152 illustrated in FIG. 6, a rectangular frame centered at the center of gravity 154 is used. Each side of the quadrangle representing the emphasis region 152 is in contact with at least any of the first region of interest 1501 or the second region of interest 1502.

That is, as the emphasis region 152 set for the first region of interest 1501 and the second region of interest 1502, a quadrangular frame including all of the first region of interest 1501 and of the second region of interest 1502 and having the smallest area may be used. The smallest quadrangular frame including all of the first region of interest 1501 and the second region of interest 1502 described in the embodiment is an example of an emphasis region including all of two or more regions of interest whose emphasis candidate regions are to be merged.

For the emphasis region 152, the shape of the first emphasis candidate region 1521 or the shape of the second emphasis candidate region 1522 may be used. For the emphasis region 152, a quadrangle including all of the first emphasis candidate region 1521 and the second emphasis candidate region 1522 may be used.

However, from the viewpoint of not hindering visual recognition of the endoscopic image 38 and from the viewpoint of not making the emphasis region 152 too large, the emphasis region 152 is preferably set in a manner illustrated in FIG. 6. The shape of the emphasis candidate region may include the concepts of the shape and the size of the emphasis candidate region.

In the present embodiment, merging of the emphasis candidate regions in the frame image 38b constituting the moving image 38a is exemplified. However, merging of the emphasis candidate regions according to the present embodiment may also be applied to the still image 39.

### First Modification

The emphasis region adjustment unit 42b may include a function of adjusting the size of the emphasis region 152. That is, the emphasis region setting unit 42 may include a size adjustment unit that adjusts the size of the emphasis region. The term “adjustment” used herein may include initial setting of the size. For example, the size of the emphasis region 152 may be adjusted in accordance with the center-of-gravity distance L between the regions of interest.

The emphasis region adjustment unit 42b may also include a function of setting the shapes of the emphasis candidate region and the emphasis region 152. That is, the emphasis region setting unit 42 may include a shape setting unit that sets the shape of the emphasis region.

### Second Modification

Instead of the center-of-gravity distance L between the regions of interest, the minimum value of a distance between edges of a plurality of regions of interest may be used as the distance between the regions of interest. That is, the emphasis region adjustment unit 42b may calculate the minimum value of the distance between the edges of the regions of interest from the coordinate values of the closed curves representing the outer shapes of the regions of interest.

### Third Modification

The emphasis region adjustment unit 42b may merge three or more emphasis candidate regions. For example, a case is considered where a third region of interest is detected in the frame image 38b illustrated in FIG. 5 and a third emphasis candidate region is set for the third region of interest.

If the center-of-gravity distance L between the first region of interest 1501 and the region of interest in the third emphasis candidate region is less than or equal to the threshold value TH, the emphasis region adjustment unit 42b merges the first emphasis candidate region 1521, the second emphasis candidate region 1522, and the third emphasis candidate region.

If the center-of-gravity distance L between the second region of interest 1502 and the region of interest in the third emphasis candidate region is less than or equal to the threshold value TH, the emphasis region adjustment unit 42b merges the first emphasis candidate region 1521, the second emphasis candidate region 1522, and the third emphasis candidate region.

On the other hand, if the center-of-gravity distance L between the first region of interest 1501 and the region of interest in the third emphasis candidate region exceeds the threshold value TH and if the center-of-gravity distance L between the second region of interest 1502 and the region of interest in the third emphasis candidate region exceeds the threshold value TH, the emphasis region adjustment unit 42b merges the first emphasis candidate region 1521 and the second emphasis candidate region 1522 but does not merge the third emphasis region with the first emphasis candidate region 1521 and the second emphasis candidate region 1522.

### Flowchart of Medical Image Processing Method

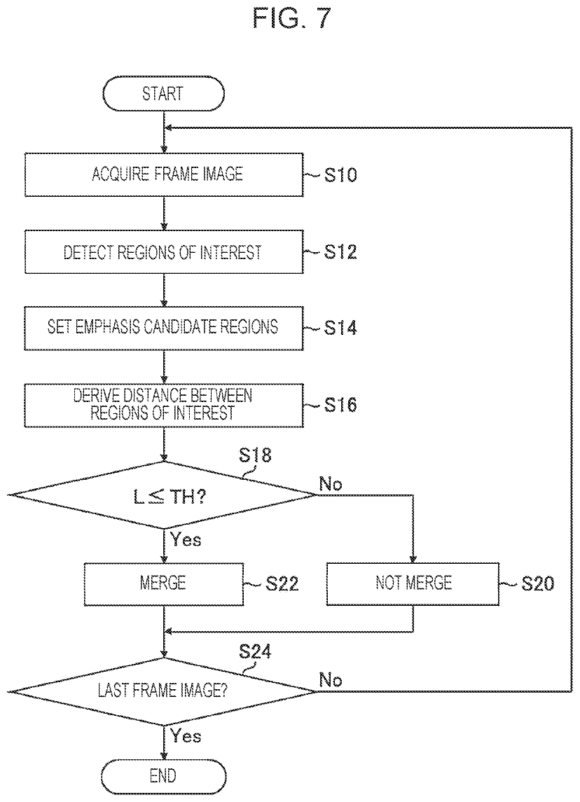

FIG. 7 is a flowchart of a medical image processing method according to the first embodiment. The medical image processing method according to the first embodiment includes an endoscopic image acquisition step S10, a region-of-interest detection step S12, an emphasis candidate region setting step S14, a region-of-interest distance derivation step S16, a merging determination step S18, a non-merging step S20, a merging step S22, and a last frame image determination step S24. The non-merging step S20 and the merging step S22 may be collectively set as an emphasis region adjustment step.

In the endoscopic image acquisition step S10, the medical image processing apparatus 14 illustrated in FIG. 3 acquires, using the image acquisition unit 40, the frame image 38b constituting the endoscopic image 38 from the endoscope system 9. The image acquisition unit 40 stores the frame image 38b in the endoscopic image storage unit 47. After the endoscopic image acquisition step S10, the process proceeds to the region-of-interest detection step S12.

In the region-of-interest detection step S12, the region-of-interest detection unit 41 detects regions of interest from the frame image 38b and identifies the location and shape of each of the regions of interest. The region-of-interest detection unit 41 stores, as information on each of the regions of interest, information on coordinate values representing the location of the region of interest and information on the shape of the region of interest in the region-of-interest storage unit 48. After the region-of-interest detection step S12, the process proceeds to the emphasis candidate region setting step S14.

Note that the region of interest used herein is a general term for a region of interest such as the first region of interest 1501 illustrated in FIG. 4 or the like. An emphasis candidate region described later is a general term for an emphasis candidate region such as the first emphasis candidate region 1521 illustrated in FIG. 4 or the like.

In the emphasis candidate region setting step S14, the emphasis candidate region setting unit 42a sets the location and shape of an emphasis candidate region on the basis of the location and shape of each of the regions of interest detected in the region-of-interest detection step S12. The emphasis candidate region setting unit 42a stores, as information on each of the emphasis candidate regions, information on the location of the emphasis candidate region and information on the shape of the emphasis candidate region in the emphasis region storage unit 49. After the emphasis candidate region setting step S14, the process proceeds to the region-of-interest distance derivation step S16.

In the region-of-interest distance derivation step S16, the emphasis region adjustment unit 42b derives the center-of-gravity distance L between the regions of interest. The emphasis region adjustment unit 42b stores the center-of-gravity distance L between the regions of interest in the emphasis region storage unit 49. After the region-of-interest distance derivation step S16, the process proceeds to the merging determination step S18.

In the merging determination step S18, the emphasis region adjustment unit 42b compares the center-of-gravity distance L between the regions of interest with the predetermined threshold value TH. A threshold value setting step may be performed as a step preceding the merging determination step S18.

If the emphasis region adjustment unit 42b determines that the center-of-gravity distance L between the regions of interest exceeds the threshold value TH, No is obtained. Thus, the process proceeds to the non-merging step S20. On the other hand, if the emphasis region adjustment unit 42b determines that the center-of-gravity distance L between the regions of interest is less than or equal to the threshold value TH, Yes is obtained. Thus, the process proceeds to the merging step S22.

In the non-merging step S20, the emphasis region adjustment unit 42b sets each of the emphasis candidate regions as the emphasis region 152 without merging the emphasis candidate regions. The emphasis region adjustment unit 42b stores information on the emphasis region 152 in the emphasis region storage unit 49. After the non-merging step S20, the process proceeds to the last frame image determination step S24.

In the merging step S22, the emphasis region adjustment unit 42b merges the plurality of emphasis candidate regions and sets the emphasis region 152. The emphasis region adjustment unit 42b stores information on the emphasis region 152 in the emphasis region storage unit 49. As a step subsequent to the merging step S22, a size adjustment step of adjusting the size of the emphasis region obtained by merging the plurality of emphasis candidate regions may be performed. As a step subsequent to the merging step S22, a shape setting step of setting the shape of the emphasis region obtained by merging the plurality of emphasis candidate regions may be performed.

As a step subsequent to the non-merging step S20 and the merging step S22, a display control step may be performed in which the display control unit 44 transmits a signal representing the regions of interest and the emphasis region 152 to the monitor device 16. After the merging step S22, the process proceeds to the last frame image determination step S24.

In the last frame image determination step S24, the image acquisition unit 40 determines whether the frame image 38b acquired in the endoscopic image acquisition step S10 is the last frame image 38b. If a period over which the next frame image 38b is not input after the acquisition of the frame image 38b is longer than or equal to a predetermined period, the image acquisition unit 40 may determine that the last frame image 38b has been acquired. The image acquisition unit 40 may determine that the last frame image 38b has been acquired, in response to receipt of a signal indicating the end of transmission of the endoscopic image 38.

In the last frame image determination step S24, if it is determined that the image acquisition unit 40 has acquired the frame image 38b that is not the last frame image 38b, No is obtained. Thus, the process proceeds to the endoscopic image acquisition step S10. Thereafter, the individual steps from the endoscopic image acquisition step S10 to the last frame image determination step S24 are repeatedly performed until Yes is obtained in the last frame image determination step S24.

On the other hand, if it is determined in the last frame image determination step S24 that the image acquisition unit 40 has acquired the last frame image 38b, Yes is obtained. After the predetermined termination processing is performed, the medical image processing method ends.

### Effects of First Embodiment

According to the medical image processing apparatus 14 of the first embodiment configured as described above, the following effects can be obtained.

[1]

When two or more regions of interest are detected, an emphasis candidate region is set for each of the regions of interest. An emphasis region obtained by merging the plurality of emphasis candidate regions is set in accordance with a distance between the regions of interest. A single emphasis region is set for the two or more regions of interest. Consequently, the plurality of emphasis candidate regions are integrated together. Thus when a region of interest in the endoscopic image 38 is reported, hindrance of visual recognition of the endoscopic image may be suppressed.

[2]

It is determined whether to merge the plurality of emphasis candidate regions using a predetermined threshold value. Consequently, a process of merging a plurality of emphasis candidate regions may be performed using a predetermined criterion.

[3]

As the distance between the regions of interest, a distance between centers of gravity of the regions of interest may be used. Consequently, the distance between the regions of interest can be derived for regions of interest having various shapes.

[4]

The emphasis region obtained by merging the plurality of emphasis candidate regions has a shape including all of the plurality of regions of interest respectively corresponding to the emphasis candidate regions. Consequently, an overlap between the region of interest and the emphasis region is avoided. Thus, hindrance of visual recognition of a plurality of regions of interest may be suppressed and the plurality of regions of interest may be emphasized.

[5]

The shape of the emphasis region obtained by merging the plurality of emphasis candidate regions is smaller than a shape obtained by adding the plurality of emphasis candidate regions. Consequently, the emphasis region obtained by merging the plurality of emphasis candidate regions may be suppressed from becoming larger than necessary.

### Medical Image Processing Apparatus According to Second Embodiment

A medical image processing apparatus according to a second embodiment will be described next. The hardware configuration illustrated in FIG. 2 and the functional blocks illustrated in FIG. 3 may be used for the medical image processing apparatus according to the second embodiment. Description of the hardware configuration and functional blocks of the medical image processing apparatus is omitted herein.

The medical image processing apparatus according to the second embodiment uses a degree of overlap of emphasis candidate regions as a distance between regions of interest. It may be determined whether to merge a plurality of emphasis candidate regions on the basis of a distance between a plurality of regions of interest even if the center-of-gravity distance L between the regions of interest described in the first embodiment is not necessarily calculated.

The degree of overlap of emphasis candidate regions is an index indicating a degree with which the plurality of emphasis candidate regions overlap. When the distance between the plurality of regions of interest becomes relatively small, the degree of overlap becomes relatively large. On the other hand, when the distance between the plurality of regions of interest becomes relatively large, the degree of overlap becomes relatively small. That is, the degree of overlap of the emphasis candidate regions may be considered as the distance between the plurality of regions of interest.

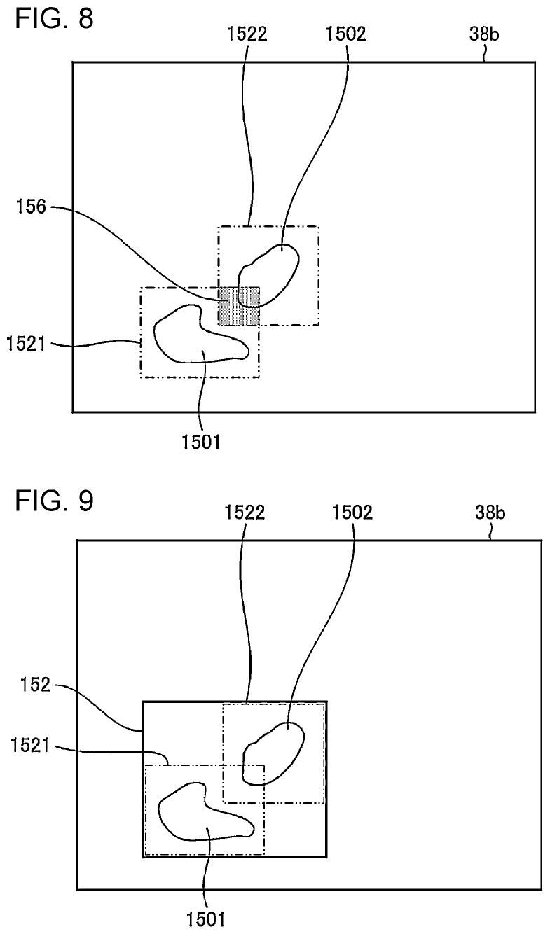

FIG. 8 is an explanatory diagram of derivation of a degree of overlap of emphasis candidate regions. The emphasis region adjustment unit 42b derives an area of an overlap region 156 of the first emphasis candidate region 1521 and the second emphasis candidate region 1522. That is, the area of the overlap region 156 is used as the degree of overlap. The emphasis region adjustment unit 42b determines whether to merge the first emphasis candidate region 1521 and the second emphasis candidate region 1522 on the basis of the area of the overlap region 156.

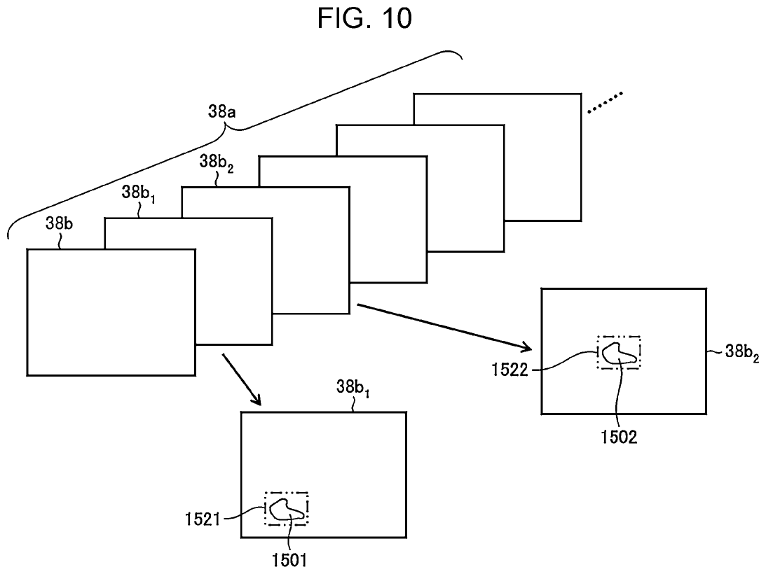

FIG. 9 is an explanatory diagram of merging of emphasis candidate regions on the basis of the degree of overlap. As illustrated in FIG. 9, if the area of the overlap region 156 illustrated in FIG. 8 is larger than or equal to a predetermined threshold value, the emphasis region adjustment unit 42b merges the first emphasis candidate region 1521 and the second emphasis candidate region 1522. The emphasis region adjustment unit 42b sets the emphasis region 152 for the first region of interest 1501 and the second region of interest 1502.

On the other hand, if the area of the overlap region 156 is smaller than the predetermined threshold value, the emphasis region adjustment unit 42b does not merge the first emphasis candidate region 1521 and the second emphasis candidate region 1522. If the area of the overlap region 156 is smaller than the predetermined threshold value, the emphasis region adjustment unit 42b sets the first emphasis candidate region 1521 as an emphasis region of the first region of interest 1501, and sets the second emphasis candidate region 1522 as an emphasis region of the second region of interest 1502.

The first modification and the third modification described in the first embodiment can be applied to the second embodiment. That is, the emphasis region adjustment unit 42b may adjust the size of the emphasis region 152 in accordance with the degree of overlap of the emphasis candidate regions. If three or more regions of interest are detected, it may be determined whether to merge emphasis candidate regions on the basis of the degree of overlap of the emphasis candidate regions and the emphasis candidate regions may be merged.

For the three or more regions of interest, a single emphasis region obtained by merging the emphasis candidate regions respectively corresponding to the regions of interest may be set.

### Effects of Second Embodiment

According to the medical image processing apparatus of the second embodiment, substantially the same effects as those of the medical image processing apparatus 14 of the first embodiment can be obtained. In addition, it may be determined whether to merge the plurality of emphasis candidate regions using the degree of overlap of the plurality of emphasis candidate regions as the distance between the plurality of regions of interest.

### Medical Image Processing Apparatus According to Third Embodiment

A medical image processing apparatus according to a third embodiment will be described next. The hardware configuration illustrated in FIG. 2 and the functional blocks illustrated in FIG. 3 may be used for the medical image processing apparatus according to the third embodiment. Description of the hardware configuration and functional blocks of the medical image processing apparatus is omitted herein.

When a plurality of regions of interest are detected across the plurality of frame images 38b constituting the moving image 38a, the medical image processing apparatus according to the third embodiment merges emphasis candidate regions respectively corresponding to the plurality of regions of interest in accordance with a distance between the plurality of regions of interest.

FIG. 10 is an explanatory diagram of merging of emphasis candidate regions used by the medical image processing apparatus according to the third embodiment. A reference sign 38b2 represents a frame of interest. The frame of interest is the frame image 38b that is focused on at any timing. A reference sign 38b1 represents the frame image 38b immediately preceding the frame of interest.

The first region of interest 1501 is detected in the frame image 38b1 illustrated in FIG. 10. The emphasis candidate region setting unit 42a sets the first emphasis candidate region 1521 for the first region of interest 1501. The second region of interest 1502 is detected in the frame image 38b₂. The emphasis candidate region setting unit 42a sets the second emphasis candidate region 1522 for the second region of interest 1502.

The emphasis candidate region setting unit 42a stores, for each frame image 38b, information on the region of interest in the region-of-interest storage unit 48. The emphasis candidate region setting unit 42a stores, for each frame image 38b, information on the emphasis candidate region in the emphasis region storage unit 49.

FIG. 10 illustrates processing performed on two frame images 38b among the plurality of frame images 38b constituting the moving image 38a. The emphasis candidate region setting unit 42a may apply the same processing to three or more frame images 38b.

The emphasis region adjustment unit 42b checks a change in the region of interest between the frame image 38b2 and the immediately preceding frame image 38b1. That is, the emphasis region adjustment unit 42b derives a distance between the second region of interest 1502 of the frame image 38b₂ and the first region of interest 1501 of the frame image 38b1.

As the distance between the first region of interest 1501 and the second region of interest 1502, the center-of-gravity distance L described in the first embodiment or the degree of overlap described in the second embodiment may be used. When the region of interest 1502 is detected in the frame image 38b2, it is probable that the region of interest 1502 at a distance close to the region of interest 1501 detected in the immediately preceding frame image 38b1 and the region of interest 1501 are the same region of interest. Accordingly, a distance from the region of interest 1502 in the frame image 38b2 is determined for the region of interest 1502 at a distance close to the region of interest 1051 detected in the immediately preceding frame image 38b1 and the region of interest 1051.

The emphasis region adjustment unit 42b may identify a plurality of emphasis candidate regions subjected to derivation of the center-of-gravity distance between the regions of interest using a first threshold value, and may determine whether to merge the plurality of emphasis candidate regions using a second threshold value that is less than the first threshold value.

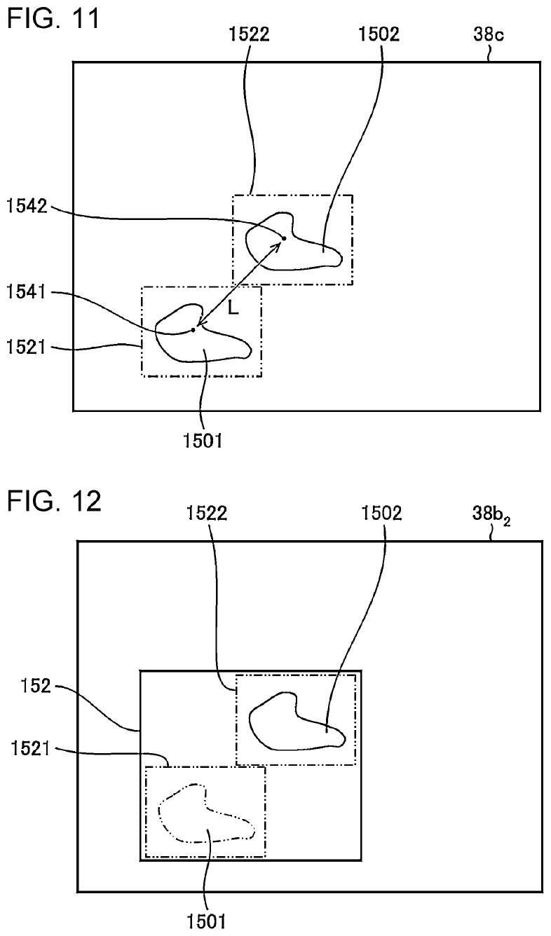

FIG. 11 is an explanatory diagram of derivation of a distance between regions of interest. FIG. 11 illustrates a virtual frame image 38c obtained by combining the frame image 38b₁ and the frame image 38b2 illustrated in FIG. 10. In the example illustrated in FIG. 11, the center-of-gravity distance L between the regions of interest is used as the distance between the regions of interest.

The emphasis region adjustment unit 42b derives the center-of-gravity distance L between the center of gravity 1541 of the first region of interest 1501 and the center of gravity 1542 of the second region of interest 1502. As in the first embodiment, coordinate values in a two-dimensional orthogonal coordinate system may be used to derive the center-of-gravity distance L.

FIG. 12 is an explanatory diagram of merging of emphasis candidate regions on the basis of the distance between regions of interest. If the center-of-gravity distance L between the center of gravity 1541 of the first region of interest 1501 and the center of gravity 1542 of the second region of interest 1502 is less than or equal to the predetermined threshold value TH, the emphasis region adjustment unit 42b merges the first emphasis candidate region 1521 and the second emphasis candidate region 1522, and sets the emphasis region 152 in the frame image 38b2.

Merging of the first emphasis candidate region 1521 and the second emphasis candidate region 1522 is as described in the first embodiment. FIG. 12 illustrates the emphasis region 152 including all of the first emphasis candidate region 1521 and the second emphasis candidate region 1522. The shape used for the emphasis region 152 obtained by merging the plurality of emphasis candidate regions is as described in the first embodiment.

### Effects of Third Embodiment

When a plurality of regions of interest are detected across the plurality of frame images 38b constituting the moving image 38a, the medical image processing apparatus of the third embodiment merges emphasis candidate regions in accordance with a distance between the regions of interest. Consequently, substantially the same effects as those of the medical image processing apparatus 14 of the first embodiment can be obtained.

As the distance between the regions of interest, the degree of overlap of the plurality of emphasis candidate regions is used. Consequently, substantially the same effects as those of the medical image processing apparatus of the second embodiment can be obtained.

If the presence or absence of the emphasis region and the location of the emphasis region change for each frame image 38b, flickering may occur in a display screen that displays the endoscopic image 38. Flickering in the display screen that displays the endoscopic image 38 may be suppressed by setting a single emphasis region for the plurality of regions of interest.

### Fourth Embodiment

A medical image processing apparatus according to a fourth embodiment will be described next. The hardware configuration illustrated in FIG. 2 and the functional blocks illustrated in FIG. 3 may be used for the medical image processing apparatus according to the fourth embodiment. Description of the hardware configuration and functional blocks of the medical image processing apparatus is omitted herein.

The medical image processing apparatus according to the fourth embodiment uses merging of a plurality of emphasis candidate regions on the basis of feature quantities of regions of interest as well.

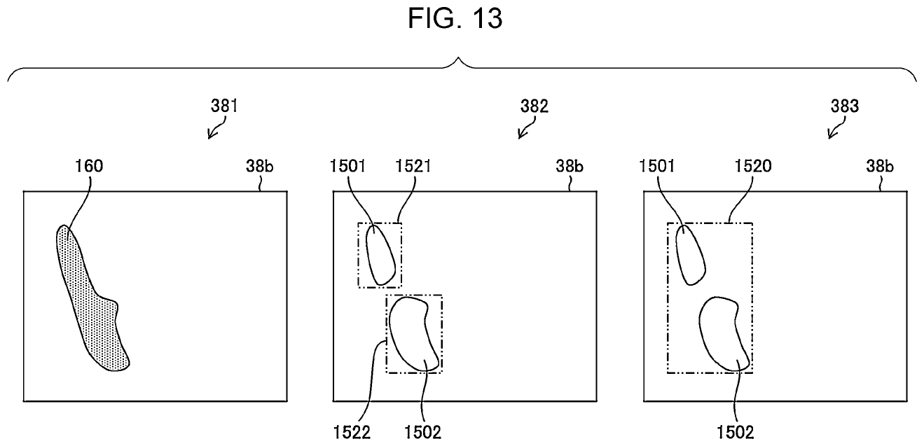

FIG. 13 is a schematic diagram of merging of a plurality of emphasis candidate regions on the basis of feature quantities of regions of interest. A reference sign 381 in FIG. 13 represents a schematic diagram of the frame image 38b in which an elongated lesion 160 is present. A reference sign 382 in FIG. 13 represents a schematic diagram of a detection result of regions of interest in the frame image 38b represented by the reference sign 381. A reference sign 383 in FIG. 13 represents a schematic diagram of the frame image 38b in which a single emphasis candidate region is set for the plurality of regions of interest on the basis of feature quantities of the plurality of regions of interest.

When the elongated lesion 160 as indicated by the reference sign 381 in FIG. 13 is present, the elongated lesion 160 cannot be detected as a single region of interest as indicated by the reference sign 382 and a plurality of regions of interest such as the first region of interest 1501 and the second region of interest 1502 indicated by the reference sign 382 may be detected.

Therefore, mucous membrane structures in the first region of interest 1501 and the second region of interest 1502 are compared with each other, and a single emphasis candidate region is set for the plurality of regions of interest with the same or similar mucous membrane structures. This enables a plurality of regions of interest to be visually recognized as a single region of interest.

The emphasis candidate region setting unit 42a derives a feature quantity of the first region of interest 1501 and a feature quantity of the second region of interest 1502, and determines whether the mucous membrane structure of the first region of interest 1501 and the mucous membrane structure of the second region of interest 1502 are the same as or similar to each other on the basis of the respective feature quantities.

If the mucous membrane structure of the first region of interest 1501 and the mucous membrane structure of the second region of interest 1502 are the same as or similar to each other, the emphasis candidate region setting unit 42a sets a single emphasis candidate region 1520 for the first region of interest 1501 and the second region of interest 1502.

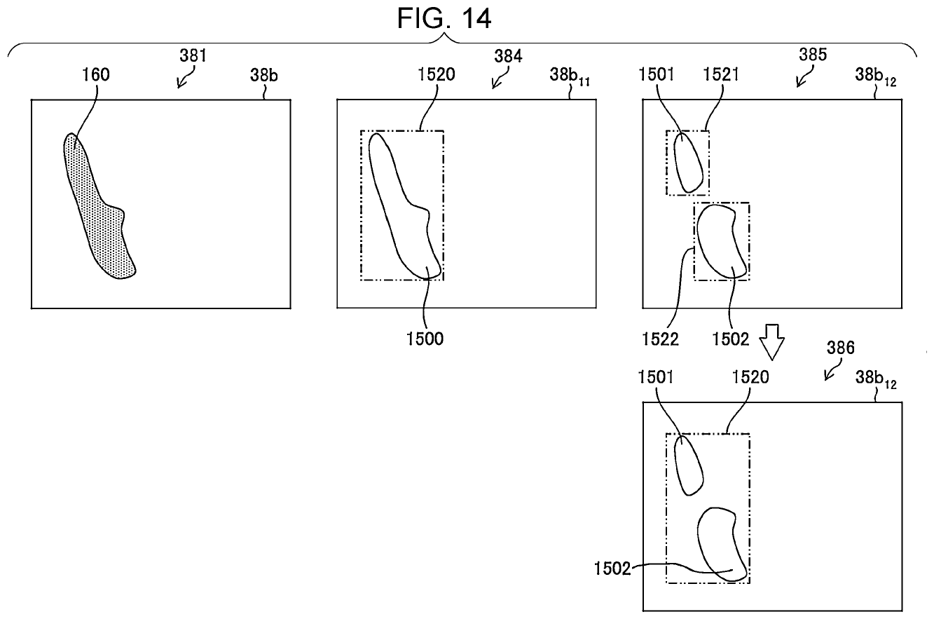

FIG. 14 is a schematic diagram of another example of merging of a plurality of emphasis candidate regions on the basis of feature quantities of regions of interest. FIG. 14 schematically illustrates merging of emphasis candidate regions when a region of interest 1500 is detected in a preceding first frame image 38b11 and the region of interest 1500 is detected as a plurality of regions of interest in a second frame image 38b12 subsequent to the first frame image 38b11.

Similarly to FIG. 13, the reference sign 381 in FIG. 14 represents a schematic diagram of the frame image 38b in which the elongated lesion 160 is present. A reference sign 384 represents a schematic diagram of the first frame image 38b11 at any timing. In the first frame image 38b11, the region of interest 1500 corresponding to the lesion 160 in the schematic diagram represented by reference sign 381 is detected. In the first frame image 38b11, the emphasis candidate region 1520 for the region of interest 1500 is set.

A reference sign 385 represents a schematic diagram of the second frame image 38b12 subsequent to the first frame image 38b11. In the second frame image 38b12, the lesion 160 detected as the region of interest 1500 in the first frame image 38b11 is detected as the first region of interest 1501 and the second region of interest 1502.

In such a case, the first emphasis candidate region 1521 for the first region of interest 1501 and the second emphasis candidate region 1522 for the second region of interest 1502 are set. However, the emphasis candidate region 1520 indicated by the reference sign 384 is supposed to be set.

Accordingly, when comparison between the feature quantity of the first region of interest 1501 and the feature quantity of the second region of interest 1502 indicates that the first region of interest 1501 and the second region of interest 1502 have the same or similar mucous membrane structure, the first emphasis candidate region 1521 and the second region of interest 1502 are merged.

In the second frame image 38b12 indicated by a reference sign 386, the first emphasis candidate region 1521 and the second emphasis candidate region 1522 are merged, and the single emphasis candidate region 1520 is set for the first region of interest 1501 and the second region of interest 1502.

### Effects of Fourth Embodiment

The medical image processing apparatus of the fourth embodiment sets a single emphasis candidate region for a plurality of regions of interest on the basis of feature quantities of the regions of interest. Consequently, the plurality of regions of interest can be handled as a single region of interest.

Flickering of the emphasis region may be suppressed, compared with the case where each of the first emphasis candidate region 1521 and the second emphasis candidate region 1522 is set as an emphasis region.

### Modifications of Endoscope System

### Modification of Processor Device

The processor device 12 may have the functions of the medical image processing apparatus 14. That is, the processor device 12 and the medical image processing apparatus 14 may be integrated together. In such an embodiment, the display device 13 may also serve as the monitor device 16. The processor device 12 may include a connection terminal to which the input device 15 is connected.

### Modification of Illumination Light

One example of the medical image acquirable using the endoscope system 9 according to the present embodiments is a normal-light image acquired by radiating light in a white range or light in a plurality of wavelength ranges as the light in the white range.

Another example of the medical image acquirable using the endoscope system 9 according to the present embodiments is an image acquired by radiating light in a specific wavelength range. A range narrower than the white range may be used as the specific wavelength range. The following modifications may be employed.

### First Modification

A first example of the specific wavelength range is a blue range or a green range in a visible range. The wavelength range of the first example includes a wavelength range of 390 nm or more and 450 nm or less or a wavelength range of 530 nm or more and 550 nm or less, and the light of the first example has a peak wavelength in the wavelength range of 390 nm or more and 450 nm or less or the wavelength range of 530 nm or more and 550 nm or less.

### Second Modification

A second example of the specific wavelength range is a red range in the visible range. The wavelength range of the second example includes a wavelength range of 585 nm or more and 615 nm or less or a wavelength range of 610 nm or more and 730 nm or less, and the light of the second example has a peak wavelength in the wavelength range of 585 nm or more and 615 nm or less or the wavelength range of 610 nm or more and 730 nm or less.

### Third Modification

A third example of the specific wavelength range includes a wavelength range in which an absorption coefficient is different between oxyhemoglobin and deoxyhemoglobin, and the light of the third example has a peak wavelength in the wavelength range in which the absorption coefficient is different between oxyhemoglobin and deoxyhemoglobin. The wavelength range of this third example includes a wavelength range of 400±10 nm, a wavelength range of 440±10 nm, a wavelength range of 470±10 nm, or a wavelength range of 600 nm or more and 750 nm or less, and the light of the third example has a peak wavelength in the wavelength range of 400±10 nm, the wavelength range of 440±10 nm, the wavelength range of 470±10 nm, or the wavelength range of 600 nm or more and 750 nm or less.

### Fourth Modification

A fourth example of the specific wavelength range is a wavelength range of excitation light that is used to observe fluorescence emitted by a fluorescent substance in a living body and excites this fluorescent substance. For example, the specific wavelength range of the fourth example is a wavelength range of 390 nm or more and 470 nm or less. Note that observation of fluorescence may be referred to as fluorescence observation.

### Fifth Modification

A fifth example of the specific wavelength range is a wavelength range of infrared light. The wavelength range of this fifth example includes a wavelength range of 790 nm or more and 820 nm or less or a wavelength range of 905 nm or more and 970 nm or less, and the light of the fifth example has a peak wavelength in the wavelength range of 790 nm or more and 820 nm or less or the wavelength range of 905 nm or more and 970 nm or less.

### Generation Example of Special-Light Image

The processor device 12 may generate a special-light image having information in the specific wavelength range on the basis of a normal-light image obtained through imaging using white light. Note that the term “generation” used herein includes “acquisition”. In this case, the processor device 12 functions as a special-light image acquisition unit. The processor device 12 obtains a signal of the specific wavelength range by performing calculation based on color information of red, green, and blue or color information of cyan, magenta, and yellow included in the normal-light image.

Note that red, green, and blue are sometimes referred to as RGB. In addition, cyan, magenta, and yellow are sometimes referred to as CMY.

### Generation Example of Feature-Quantity Image

As the medical image, a feature-quantity image may be generated by using calculation based on at least any of a normal-light image obtained by radiating light in the white range or light in a plurality of wavelength ranges as the light in the white range or a special-light image obtained by radiating light in the specific wavelength range. Application Example to Program for Causing Computer to Function as Image Processing Apparatus

The above-described medical image processing method can be configured as a program that implements functions corresponding to respective steps of the medical image processing method using a computer. For example, a program may be configured to cause a computer to implement an image acquisition function of acquiring a medical image; a region-of-interest detection function of detecting regions of interest from the medical image; an emphasis candidate region setting function of setting, for each of the regions of interest, an emphasis candidate region that is a candidate for an emphasis region for emphasizing the region of interest when the medical image is displayed using a display device; an emphasis region adjustment function of setting, in a case where two or more regions of interest are detected, the emphasis region obtained by merging the emphasis candidate regions respectively corresponding to the two or more regions of interest in accordance with a distance between the two or more regions of interest; and a display control function of causing the display device to display an emphasis region obtained by merging the emphasis candidate regions respectively corresponding to the two or more regions of interest.

A program that causes a computer to implement the above-described image processing function may be stored on a computer-readable information storage medium which is a non-transitory tangible information storage medium, and the program may be provided using the information storage medium.

In addition, instead of the configuration in which the program is stored on a non-transitory information storage medium and is provided, a configuration in which a program signal is provided via a network may be employed.

### Combination of Embodiments, Modifications, Etc.

The constituent elements described in the embodiments above and the constituent elements described in the modifications can be appropriately used in combination, and some of the constituent elements can be replaced.

In the embodiments of the present invention described above, the constituent elements can be appropriately changed, added, or deleted within a scope not departing from the gist of the present invention. The present invention is not limited to the embodiments described above, and various modifications can be made by a person having the ordinary skill in the art within the technical sprit of the present invention.

### REFERENCE SIGNS LIST

9 endoscope system
10 endoscope
11 light source device
12 processor device
13 display device
14 medical image processing apparatus
15 input device
16 monitor device
20 insertion section
21 operation section
22 universal cord
25 soft part
26 bending part
27 tip part
27a tip surface
28 imaging element
29 bending operation knob
30 air/water supply button
31 suction button
32 still image capturing instruction part
33 treatment tool introduction port
35 light guide
36 signal cable
37a connector
37b connector
38 endoscopic image
38a moving image
38b frame image
38b₁ frame image
38b₂ frame image
38b11 first frame image
38b12 second frame image
38c frame image
39 still image
40 image acquisition unit
41 region-of-interest detection unit
42 emphasis region setting unit
42a emphasis candidate region setting unit
42b emphasis region adjustment unit
44 display control unit
46 storage unit
47 endoscopic image storage unit
48 region-of-interest storage unit
49 emphasis region storage unit
120 control unit
122 memory
124 storage device
126 network controller
128 power supply device
130 display controller
132 input/output interface
134 input controller
136 bus
140 network
152 emphasis region
154 center of gravity
156 overlap region
160 lesion
381 reference sign
382 reference sign
383 reference sign
384 reference sign
385 reference sign
386 reference sign
1500 region of interest
1501 first region of interest
1502 second region of interest
1520 emphasis candidate region
1521 first emphasis candidate region
1522 second emphasis candidate region
1541 center of gravity
1542 center of gravity
L center-of-gravity distance
S10 to S24 steps of medical image processing method

What is claimed is:

1. A medical image processing apparatus comprising:
an image acquisition unit that acquires a medical image;
a region-of-interest detection unit that detects regions of interest from the medical image;
an emphasis candidate region setting unit that sets, for each of the regions of interest, an emphasis candidate region that is a candidate for an emphasis region for emphasizing the region of interest when the medical image is displayed using a display device;
an emphasis region adjustment unit that sets, in a case where two or more regions of interest are detected, the emphasis region obtained by merging the emphasis candidate regions respectively corresponding to the two or more regions of interest in accordance with a distance between the two or more regions of interest; and
a display control unit that causes the display device to display the emphasis region, wherein
the image acquisition unit acquires a moving image as the medical image, and
in a case where two or more regions of interest are detected in two or more different frame images constituting the moving image, the emphasis region adjustment unit merges the emphasis candidate regions respectively corresponding to the two or more regions of interest in accordance with a distance between the two or more regions of interest.

2. The medical image processing apparatus according to claim 1, wherein
the image acquisition unit acquires a still image as the medical image, and
in a case where two or more regions of interest are detected in the still image, the emphasis region adjustment unit merges the emphasis candidate regions respectively corresponding to the two or more regions of interest in accordance with a distance between the two or more regions of interest.

3. The medical image processing apparatus according to claim 1, wherein in a case where a region of interest detected as a single region of interest in a first frame image is detected as two or more regions of interest in a second frame image subsequent to the first frame image, the emphasis region adjustment unit merges the emphasis candidate regions respectively corresponding to the two or more regions of interest.

4. The medical image processing apparatus according to claim 2, wherein the emphasis region adjustment unit merges the emphasis candidate regions respectively corresponding to the two or more regions of interest in a case where the distance between the two or more regions of interest is greater than 0 and is less than or equal to a predetermined threshold value.

5. The medical image processing apparatus according to claim 1, wherein the emphasis region adjustment unit merges the emphasis candidate regions respectively corresponding to the two or more regions of interest in accordance with a distance between centers of gravity of the two or more regions of interest.

6. The medical image processing apparatus according to claim 1, wherein the emphasis region adjustment unit merges the emphasis candidate regions respectively corresponding to the two or more regions of interest in accordance with a degree of overlap of the emphasis candidate regions respectively corresponding to the two or more regions of interest.

7. The medical image processing apparatus according to claim 1, wherein the emphasis region adjustment unit merges the emphasis candidate regions respectively corresponding to the two or more regions of interest in accordance with feature quantities of the regions of interest.

8. The medical image processing apparatus according to claim 1, wherein the emphasis region adjustment unit sets the emphasis region that includes all of the two or more regions of interest to be merged.

9. A processor device comprising:
an endoscope control unit that controls an endoscope apparatus;
an image acquisition unit that acquires a medical image from the endoscope apparatus;
a region-of-interest detection unit that detects regions of interest from the medical image;
an emphasis candidate region setting unit that sets, for each of the regions of interest, an emphasis candidate region that is a candidate for an emphasis region for emphasizing the region of interest when the medical image is displayed using a display device;
an emphasis region adjustment unit that sets, in a case where two or more regions of interest are detected, the emphasis region obtained by merging the emphasis candidate regions respectively corresponding to the two or more regions of interest in accordance with a distance between the two or more regions of interest; and
a display control unit that causes the display device to display the emphasis region, wherein
the image acquisition unit acquires a moving image as the medical image, and
in a case where two or more regions of interest are detected in two or more different frame images constituting the moving image, the emphasis region adjustment unit merges the emphasis candidate regions respectively corresponding to the two or more regions of interest in accordance with a distance between the two or more regions of interest.

10. A medical image processing method comprising:
an image acquisition step of acquiring a medical image;
a region-of-interest detection step of detecting regions of interest from the medical image;
an emphasis candidate region setting step of setting, for each of the regions of interest, an emphasis candidate region that is a candidate for an emphasis region for emphasizing the region of interest when the medical image is displayed using a display device;
an emphasis region adjustment step of setting, in a case where two or more regions of interest are detected, the emphasis region obtained by merging the emphasis candidate regions respectively corresponding to the two or more regions of interest in accordance with a distance between the two or more regions of interest; and
a display control step of causing the display device to display the emphasis region, wherein
in the image acquisition step, a moving image is acquired as the medical image, and
in the emphasis region adjustment step, in a case where two or more regions of interest are detected in two or more different frame images constituting the moving image, the emphasis candidate regions respectively corresponding to the two or more regions of interest are merged in accordance with a distance between the two or more regions of interest.

11. A non-transitory computer-readable storage medium storing instructions that, when read by a computer, cause the computer to execute:
an image acquisition function of acquiring a medical image;
a region-of-interest detection function of detecting regions of interest from the medical image;
an emphasis candidate region setting function of setting, for each of the regions of interest, an emphasis candidate region that is a candidate for an emphasis region for emphasizing the region of interest when the medical image is displayed using a display device;
an emphasis region adjustment function of setting, in a case where two or more regions of interest are detected, the emphasis region obtained by merging the emphasis candidate regions respectively corresponding to the two or more regions of interest in accordance with a distance between the two or more regions of interest; and
a display control function of causing the display device to display the emphasis region, wherein
in the image acquisition function, a moving image is acquired as the medical image, and
in the emphasis region adjustment function, in a case where two or more regions of interest are detected in two or more different frame images constituting the moving image, the emphasis candidate regions respectively corresponding to the two or more regions of interest are merged in accordance with a distance between the two or more regions of interest.
